(19) United States
(12) Patent Application Publication
(10) Pub. No.: US 2024/0125949 A1
(43) Pub. Date: Apr. 18, 2024

(54) METHOD FOR MANUFACTURING A RADIATION DETECTOR MODULE AND RADIATION DETECTOR MODULE

(71) Applicant: Siemens Healthcare GmbH, Erlangen (DE)

(72) Inventor: Harald Geyer, Bubenreuth (DE)

(21) Appl. No.: 18/489,455

(22) Filed: Oct. 18, 2023

(30) Foreign Application Priority Data
Oct. 18, 2022 (DE) 10 2022 211 038.2

(51) Int. Cl. G01T 1/20; G01T 1/29

(52) U.S. Cl. CPC G01T 1/20188; G01T 1/20182; G01T 1/2985

(57) ABSTRACT

A method for manufacturing a radiation detector module including a sensor component and a heat dissipation component includes arranging a reactive multilayer system between the sensor component and the heat dissipation component. The sensor component and the heat dissipation component are brought together. The reactive multilayer system is activated for creating an RMS connection between the sensor component and the heat dissipation component. A radiation detector module manufactured with this method, as well as a radiation detector with such radiation detector modules and an imaging system, and also a replacement part for a radiation detector module are also provided.

This application claims the benefit of German Patent Application No. DE 10 2022 211 038.2, filed on Oct. 18, 2022, which is hereby incorporated by reference in its entirety.

### BACKGROUND

The present embodiments relate to a method for manufacturing a radiation detector module and to a radiation detector module manufactured with this method, as well as to a radiation detector with such radiation detector modules and to an imaging system. The present embodiments also relate to a replacement part for a radiation detector module.

Radiation detectors are employed in many imaging applications. Radiation detectors (e.g., x-ray detectors) are thus used, for example, in computed tomographs in medical imaging in order to generate a tomographic x-ray image of an examination area of a patient.

Counting direct-converting detectors or integrating indirect-converting detectors may be used in imaging (e.g., in computed tomography, angiography, or radiography). The x-ray radiation or the photons may be converted into electrical pulses by a suitable converter material in direct converting detectors. CdTe, Cadmium zinc telluride (CZT), CdZnTeSe, CdTe—Se, CdMnTe, InP, TlBr2, HgI2, GaAs, or another material may be used as converter material, for example. The electrical pulses may be evaluated by electronic circuits of an evaluation unit, for example, in the form of an integrated circuit (e.g., Application Specific Integrated Circuit (ASIC)). In counting detectors, the incident x-ray radiation may be measured by counting the electrical pulses that are triggered by the absorption of x-ray photons in the converter material. Further, the height of the electrical pulse is as a rule proportional to the energy of the absorbed x-ray photon. This enables spectral information to be extracted by a comparison of the height of the electrical pulse with a threshold value. In indirect converting detectors, the x-ray radiation or the photons may be converted into light by a suitable converter material and be converted by optically-coupled photodiodes into electrical pulses. Scintillators (e.g., GOS (Gd2O2S), CsJ, YGO or LuTAG) are frequently employed as the converter material. The electrical signals generated are further processed via an evaluation unit having electronic circuits.

A fundamental challenge in building a radiation detector lies in the dissipation of the heat arising, both in the converter itself and also in the waste heat that arises in the evaluation units. The waste heat of an individual evaluation unit or sensor may lie in the order of magnitude of 1-2 Watts, for example. Frequently, heat is removed by complete cooling of the detector by blowing in cooled air.

For effective dissipation of waste heat by a flow of air, the structure described above is generally connected to a heat sink. Via a thermal bonding of the heat sink to the functional part of the sensor (e.g., using a thermally conducting paste or an adhesive able to conduct heat), the waste heat is dissipated from the sensor or ASIC into the metallic heat sink (e.g., in most cases aluminum) to the flow of air.

The thermal bonding itself may provide that the heat sink is attached or that there is additional adhesion (e.g., by UV glue points) that may also represent a pre-fixing for an adhesive capable of conducting heat.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for manufacturing a radiation detector module and a radiation detector module manufactured in this way that overcome the disadvantages of the prior art and allow a good heat dissipation, with manufacturing that is as protective as possible, are provided. As another example, a radiation detector with such radiation detector modules and an imaging system (e.g., a computed tomography system, an angiography system, a radiography system, or a system for mammo tomosynthesis) are provided. As yet another example, a replacement part for a radiation detector module is provided.

A method of the present embodiments is used for manufacturing a radiation detector module. The radiation detector module includes a sensor component and a heat dissipation component. The sensor component in this case is a functional component for measurement and typically includes a converter unit for conversion of radiation into electrical signals (e.g., a scintillator and a photodiode), often an intermediate layer, and a number of evaluation units (e.g., ASICS). The heat dissipation component has a comparatively large surface that may be used for heat dissipation (e.g., by radiation or dissipation to a liquid or to a flow of air). In one embodiment, the heat dissipation component is a heat sink or a carrier unit. The carrier unit may include circuit board material and electrical lines (e.g., conductor tracks) for conveying data from the number of evaluation units and for supply of energy.

The method includes the following acts: arrangement of a reactive multilayer system (RMS) between the sensor component and the heat dissipation component; bringing together (e.g., pressing together) of the sensor component and the heat dissipation component; and activating the reactive multilayer system for creating an RMS connection between the sensor component and the heat dissipation component.

Reactive multilayer systems (RMS) are known in the prior art and include two or more alternating thin metallic films or layers. In such systems, the reactive multilayer systems alternate between a layer that is referred to as the “reactive layer” (e.g., containing aluminum, titanium, nickel, or a-silicon) and a layer that is referred to as the “solder layer” (e.g., containing gold, copper, aluminum, titanium, or metallic glass). The materials are to differ from one another in such cases and are to be selected so that, on activation by an electrical pulse, by pressure, by an increase in temperature, or by a laser pulse, a self-propagating exothermic reaction takes place within the multilayer system. Because of the low temperature to which the surrounding material is exposed, temperature-sensitive components and materials such as, for example, metals, polymers, and ceramics may be joined together for conduction of heat without thermal damage. The heat arising is localized at the bonding border surface and limited by a brief heating up phase within milliseconds. This low heat is an advantage. The low heat prevents sensor components, which will only withstand a little heat (e.g., zinc telluride or cadmium telluride), from being subjected to any high temperatures and makes rapid cooling possible. The thickness of the layers lies in the nanometer range.

The arrangement of the reactive multilayer system may be a very simple design, for example, in the form of a cut or punched shape from film (e.g., many RMS s are activated at much higher pressures) that is laid between the sensor component and the heat dissipation component, but is also very precise and structured (e.g., by sputtering or etching of the layers directly onto the sensor component and/or heat dissipation component).

For a good connection, the sensor component and the heat dissipation component are to be brought together with the RMS lying between the sensor component and the heat dissipation component in the position in which the sensor component and the heat dissipation component are later to be connected with one another. In one embodiment, for a connection that is as firm as possible, the sensor component and the heat dissipation component are pressed (e.g., lightly) against one another, but the pressure is to be configured so that none of the components sustain damage. Basically, it is sufficient for the sensor component with intermediate RMS to be laid on the heat dissipation component and to remain there without moving. There may be a pre-fixing of the components (e.g., by UV glue points).

As already indicated above, the activation may be by an increase in temperature, a pressure, or a laser pulse. All these methods may, however, damage the sensor component thermally or mechanically. The activation may, therefore, be undertaken by a current pulse. To do this, a current is simply applied to two points on one side of the RMS, so that a reaction is initiated there. Since an exothermic reaction is involved, the RMS will then react from this point outwards until, instead of the RMS, an RMS connection is present. Even if, in the RMS connection, the layers of the RMS have reacted with one another, it may be established in a cross sectional image under an electron microscope or in a spectroscopic examination that the RMS connection differs from a conventional glued connection. The RMS connection thus serves both to fasten the sensor component to the heat dissipation component and also to create a thermal contact between these components.

In one embodiment, the radiation detector module was manufactured with the method of the present embodiments and includes a sensor component and a heat dissipation component that are connected to one another by the RMS connection.

In one embodiment, a radiation detector includes a plurality of radiation detector modules of the present embodiments arranged next to one another. These may indeed be attached to a common heat sink or a common carrier unit by the RMS connection.

In one embodiment, an imaging system (e.g., a medical imaging system) is, for example, a CT system, an angiography system, a mammography system (or mammo tomosynthesis system), or a radiography system and includes a radiation detector of the present embodiments (and thus also a plurality of radiation detector modules of the present embodiments) and a radiation source positioned opposite the radiation detector. The radiation source is configured to irradiate the radiation detector. The advantages of the imaging system of the present embodiments essentially correspond to the advantages of the radiation detector modules. Features, advantages, or alternate forms of embodiment mentioned here may likewise also be transferred to the imaging system and vice versa.

The imaging system may be configured as a computed tomography device. In other embodiments, the imaging system may also be another type of imaging system and, for example, may be an imaging system based on x-ray radiation. The radiation source may then, for example, be configured as an x-ray source, where the radiation detector is configured to detect x-ray radiation. For example, the imaging system may be a C-arm x-ray device, a mammography device, or an x-ray imaging system for radiography.

In one embodiment, a replacement part for a radiation detector module of the present embodiments (e.g., also in a radiation detector of the present embodiments or in an imaging system of the present embodiments) includes a sensor component and a reactive multilayer system on the side of the sensor component that is configured to be connected to a heat dissipation component of the radiation detector module. The reactive multilayer system may, for example, be fixed to the sensor component by a number of glue points.

In the case of a radiation detector in which a number of radiation detector modules are accommodated on a common heat sink, a damaged radiation detector module may simply be released, any possible residues of its RMS connection removed, and the replacement part simply laid on the heat sink and then activated. This enables the replacement part to be connected to the heat sink for heat conduction and produces a radiation detector module of the preset embodiments. This enables a radiation detector to be repaired in a simple way.

Further, embodiments and developments emerge from the dependent claims and from the description given below. The claims of one claim category may also be developed by analogy with the claims and description parts into another claim category and, for example, individual features of different embodiments or variants may be combined to form new embodiments or variants.

In accordance with a form of embodiment, the heat dissipation component is a heat sink, and the sensor component includes a carrier unit (e.g., a printed circuit board), on which conductor tracks for conveying signals and for energy supply are arranged. The carrier unit may have a number of solid material cores that extend through the thickness of the carrier unit and represent a heat channel between upper side and lower side of the carrier material. The reactive multilayer system may be arranged at least between the heat sink and the carrier unit so that, after this activation, the heat sink and the carrier unit are connected to one another by the RMS connection. Depending on application, the reactive multilayer system may be arranged between an evaluation unit and the carrier unit, so that, after its activation, the evaluation unit and the carrier unit are connected to one another by the RMS connection. The reactive multilayer system may be attached at least between the heat sink and the carrier unit, so that, after its activation, the heat sink and the carrier unit are connected to one another by the RMS connection.

The heat sink may include a metal (e.g., aluminum). A metal may provide good thermal conductivity. Aluminum is, for example, relatively light and thereby makes it possible to keep the weight of the radiation detector modules low. A structure (e.g., a carrier structure) of a detector or of an imaging system may also readily be used as a heat sink.

In accordance with a form of embodiment, the reactive multilayer system (RMS) is attached to the heat sink or the carrier unit as an alternating arrangement of lateral layers (e.g., by sputtering, electrochemical deposition (galvanization), or etching). In one embodiment, the RMS is attached to the carrier unit side to which the heat sink is to be attached, and/or to the side on which an evaluation unit is to be attached. Attaching the RMS to the carrier unit has the advantage that the carrier unit is quite insensitive to heat, current, and chemical processes and is very easy to handle. This type of production has the advantage that complex structures may be produced.

In accordance with an alternative form of embodiment, the reactive multilayer system is taken from a film consisting of reactive multilayer material (e.g., cut out or punched out), and arranged between sensor component and heat dissipation component. In this case, the film may be pre-fixed to at least one of the components, so that the film does not slip. This may be undertaken, for example, with a simple glue point. As regards an activation by pressure, the activation energy of RMS films is typically high enough for punching or cutting to be readily possible. This type of production has the advantage of being very simple and low-cost.

A reactive multilayer system of the present embodiments is constructed of alternating solder layers and reactive layers, of which the materials differ. The solder layers may include a number of materials from the group gold, copper, aluminum, titanium, and metallic glass, and/or the reactive layers may include a material from the group aluminum, titanium, nickel, a-silicon (amorphous Si), and cobalt.

In accordance with a form of embodiment, conductor tracks are present in the sensor component and/or the heat dissipation component, or on the reactive multilayer system. The conductor tracks extend at least to the edge of the sensor component and/or heat dissipation component. Using the conductor tracks, a voltage is applied for activation of the reactive multilayer system. In practice, it is of advantage for these conductor tracks to be formed on the carrier unit, since conductor tracks are typically present there in any event. Alternatively, the conductor tracks may be formed by the reactive multilayer system (e.g., when this is formed by an RMS film). In one embodiment, parts of the conductor tracks that project over the sensor component and/or the heat dissipation component may be disconnected after activation. These parts are no longer needed after activation and may cause problems.

In one embodiment, a radiation detector module includes a stack arrangement made up of a detection layer with a number of converter units configured to convert incident radiation into electrical signals, a number of evaluation units configured to evaluate the electrical signals fed in by the detection layer, and, for example, additionally a carrier unit. This carrier unit may readily also be seen as a heat dissipation component if the carrier unit is to perform this function.

The radiation detector module may be configured for detection of x-ray radiation. In one embodiment, the radiation detector module is configured to be employed in a medical detector (e.g., for a computed tomography system, an angiography system, a radiography system, or a system for mammo tomosynthesis).

In one embodiment of a radiation detector module, the sensor component also includes a carrier unit. The number of evaluation units in the stack arrangement is arranged between the detection layer and the carrier unit. In the carrier unit, in a flat area that corresponds to a projection of one respective evaluation unit of the number of evaluation units in the stack direction, one solid material core made of a thermally-conductive material may be employed. This solid material core extends in this case, for example, over the greater part of the respective surface area and may be in contact for heat conduction via a thermally-conductive filler material (e.g., by an RMS connection) to the respective evaluation unit.

In accordance with one embodiment of a radiation detector module, the solid material core extends from the upper side of the carrier unit to the lower side of the carrier unit. The upper side of the carrier unit faces towards the number of evaluation units in the stack arrangement, and the lower side of carrier unit faces away from the number of evaluation units. In one embodiment, at least one evaluation unit is attached by the RMS connection to a solid material core. The solid material core may include a metal or a thermally conducting ceramic. The solid material core represents an especially large contact surface and through its embodiment as a massive solid material core with a low thermal resistance, an especially effective option for dissipating the heat generated in the evaluation unit or in the converter unit by the carrier unit.

One embodiment of a radiation detector module includes a heat sink that is attached by the RMS connection to the carrier unit. In one embodiment, a solid material core of the carrier unit is contacted by the RMS connection to the heat sink. This creates an advantageous thermal connection to the heat sink. The carrier unit may include circuit board material, a ceramic, a glass, or a composite material. The solid material core may be pressed into the carrier unit.

In one embodiment, the sensor unit includes direct converting detectors (also referred to as photon counting detectors). These convert the radiation falling onto them (e.g., in a semiconductor material) directly into an electrical signal. Detectors of this type include, as their preferred converter element, a converter unit made of Si (silicon), GaAs (Gallium arsenide), HgI₂ (mercury iodide), and/or a-Se (amorphous selenium) (e.g., made of CdTe (cadmium telluride) and/or CdZnTe (cadmium zinc telluride)). The latter, for example, are very sensitive to heat, so that the RMS connection offers great benefits here.

The radiation detector module includes a plurality of pixel elements (e.g., the smallest surface areas within the detection layer that may be read out individually). In order to be read out, each pixel is connected to an assigned evaluation pixel element of an evaluation unit. In this case, a number of pixel elements may be connected to an evaluation unit. The evaluation unit serves in general to digitize the electronic signals that are fed in from the number of converter units. They may be implemented for example as an Application Specific Integrated Circuit (ASIC). In them, for example, in direct converting detectors, the electronic signals detected at the respective pixels are amplified as pulses, shaped and counted or suppressed, depending on pulse height and threshold value. In such cases, heat arises in the readout units, which may be dissipated effectively by an RMS connection to a heat sink or a carrier material.

### BRIEF DESCRIPTION OF THE DRAWINGS

Same components are labeled with same reference characters in the various figures. The figures are as a rule not true-to-scale. In the figures:

FIG. 1 shows an example of a form of embodiment of an imaging system;

FIG. 2 shows a radiation detector module in accordance with the prior art;

FIG. 3 shows a radiation detector module in accordance with the prior art;

FIG. 4 shows a plan view of a layer of thermally conductive filler material in accordance with the prior art;

FIG. 5 shows an example of a form of embodiment of a radiation detector module;

FIG. 6 shows a plan view of an example of an RMS connection; and

FIG. 7 shows an execution sequence for manufacturing of an embodiment of a radiation detector module.

### DETAILED DESCRIPTION

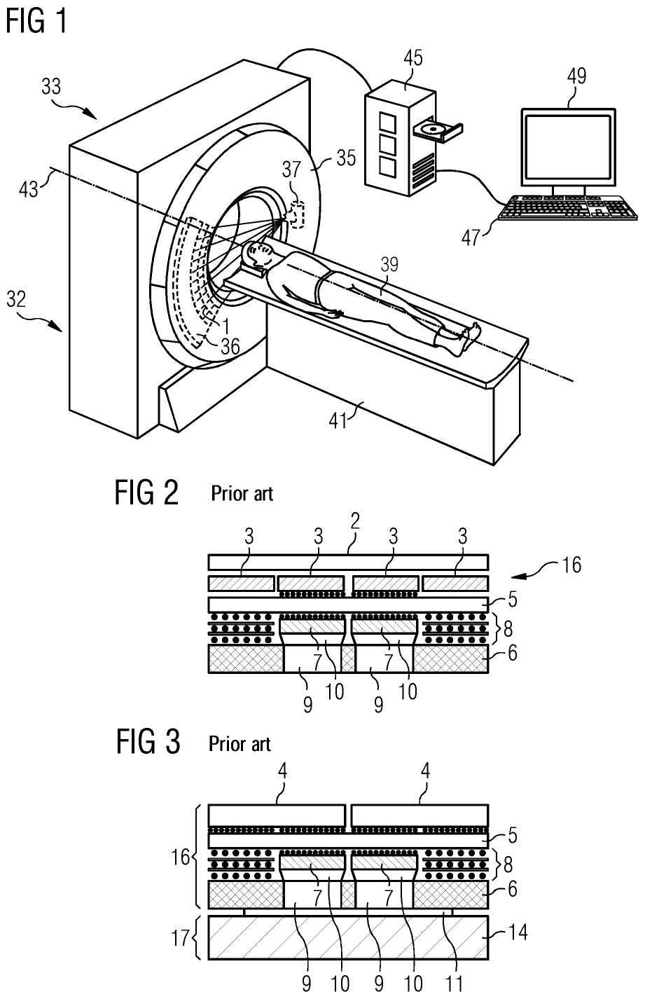

FIG. 1 shows an example of a form of embodiment of an imaging system 32 (e.g., a medical imaging system) with a radiation detector 36 including at least one radiation detector module 1 of the present embodiments and a radiation source 37 opposite the radiation detector 36. The radiation source 37 is configured to illuminate the radiation detector 36 with radiation. The medical imaging system 32 shown is configured, in this example, as a computed tomography device. The computed tomography device includes a gantry 33 with a rotor 35. The rotor 35 includes an x-ray source as the radiation source 37, and the radiation detector 36, which is configured to detect x-ray radiation.

The rotor 35 is rotatable about an axis of rotation 43. An examination object 39 (e.g., a patient) is supported on a patient couch 41 and is able to be moved along the axis of rotation 43 through the gantry 33. In general, the object 39 may include a human or animal patient, for example. A processing unit 45 is provided for control of the imaging system 32 and/or for creation of the x-ray image dataset based on signals detected by the radiation detector 36.

In the case of a computed tomography device, an x-ray image dataset (e.g., a raw x-ray image dataset) of the object 39 may be recorded by the radiation detector 36 from a plurality of angular directions that are based on processed electrical pixel measurement signals of the pixel electronics 6 of the evaluation units. Subsequently, based on the (raw) x-ray image dataset, a final x-ray image dataset is reconstructed by a mathematical method (e.g., including a filtered back projection or an iterative reconstruction method).

The radiation detector 36 includes a plurality of radiation detector modules 1 of the present embodiments. The plurality of radiation detector modules 1 are arranged next to one another in at least one direction (e.g., a direction of rotation) in order to provide a large detection surface. The plurality of radiation detector modules 1 may also be arranged, for example, next to one another in a second direction as well (e.g., along the axis of rotation 43).

The processing unit 45 (e.g., formed by one or more processors) may include a control unit for controlling the imaging system 32 and a generation unit for generation of an x-ray image dataset based on the pixel measurement signals.

Further, an input facility 47 and an output facility 49 are connected to the processing unit 45. The input facility 47 and the output facility 49 may make an interaction by a user or display of a generated x-ray image dataset possible, for example.

FIGS. 2 and 3 show radiation detector modules in accordance with the prior that are configured for detection of x-ray radiation. The radiation detector modules shown each include a stack arrangement. The stack arrangement includes a detection layer with a number of converter units 2, 3, 4 configured to convert radiation arriving at the number of converter units 2, 3, 4 into electrical signals, a number of evaluation units 7 (e.g., ASICs) configured to evaluate the electrical signals fed in from the detection layer, and a carrier unit 6.

The arrangement in a stack arrangement includes the radiation detector module being arranged in a stack direction essentially in layers. The stack direction may, for example, essentially correspond to an incident radiation direction when the radiation detector module is being irradiated with radiation.

The number of evaluation units 7 is arranged in the stack arrangement between the detection layer and the carrier unit 6. For example, the number of evaluation units 7, in the examples shown, corresponds to a plurality of evaluation units 7. In the cross-sectional view, two evaluation units 7 are shown by way of example. However, the number may include more than two (e.g., four or eight). Further, in the carrier unit 6, in a surface area that corresponds to a projection of a respective evaluation unit 7 of the number of evaluation units 7 in the stack direction, a solid material core 9 made of a thermally conducting material is employed. The solid material core 9 extends over the greater part of the respective surface area and is in contact for thermal conduction, via a thermally conducting filler material 10, with the respective evaluation unit 7.

FIG. 2 in this case shows a radiation detector module in the form of an indirect converting, integrating detector module. The detection layer includes, for example, a scintillator 2 that is coupled to photodiodes grouped into modules. A converter unit configured to convert radiation arriving at the converter unit into electrical signals is correspondingly formed, for example, from one of the modules and the surface area of the scintillator 2 assigned to the one module.

FIG. 3 by contrast shows a radiation detector module in the form of a direct converting, photon counting detector module. In this case, the detection layer includes a number of converter units 4 (e.g., semiconductor converter units). The converter units 4 are configured to convert x-ray radiation arriving at the converter units 4 directly into electrical signals. The number of converter units 4 shown in this case is purely by way of example. More or fewer converter units 4 may also be included.

The radiation detector module in both types includes a plurality of pixel elements in each case (e.g., the smallest surface areas within the detection layer that may be read out individually). In order to be read out, each pixel element of the detection layer is connected to an assigned evaluation pixel element of an evaluation unit 7, in which an evaluation and digitization of the electronic signals takes place. The evaluation units 7 may be implemented as ASICs, for example.

In accordance with a form of embodiment, an intermediate layer 5 is configured, in all forms of embodiment shown in each case, between the number of evaluation units 7 and the detection layer. The intermediate layer 5 has a plurality of electrically conducting connections between the detection layer and the number of evaluation units 7.

The electrical signals from the converter units 2, 3, 4 are forwarded via solder connections to the intermediate layer 5 and via the electrically conducting connections contained therein, and likewise, via solder connection provided for this purpose, to the evaluation units 7.

Using an intermediate layer 5, a spatial arrangement of the pixels of the detection layer may be achieved from the spatial arrangement of the evaluation pixel elements in the evaluation units 7. The electrically conducting connections in the intermediate layer 5 may be configured for this as through-contacting elements and interposer structures. The intermediate layer 5 may, for example, include a substrate made of a glass fiber composite material, circuit board material, synthetic resin bonded paper, ceramic, and/or glass.

Further, a conducting support structure 8, including a number of elements for forwarding of data from the number of evaluation units 7 to the carrier unit 6, is arranged in each case between the intermediate layer 5 and the carrier unit 6. These are configured as, for example, ballstack structures 8. The ballstack structures 8 are a layered arrangement of parallel circuit boards and solder balls arranged between the parallel circuit boards. The solder balls connect the parallel circuit boards to one another. The parallel circuit boards may have interposer structures configured using usual methods. Through-mold vias in a mold material may represent another possibility for implementing the support structures, for example.

The evaluation units 7 have outputs that are likewise connected via the intermediate layer 5 to the ballstack structures 8 and via which data may be forwarded from the number of evaluation units 7.

The carrier unit 6 may have circuit board material and likewise conductor tracks that are conductively connected to the ballstack structures 8. The conductor tracks of the carrier unit 6 may then be routed to a connector. Via these, the radiation detector of the present embodiments may be connected to further processing units (not shown here) such as, for example, an evaluation computer or a reconstruction facility of a CT device.

The solid material core 9 in the carrier unit may include a metal (e.g., aluminum or copper) or a thermally conducting ceramic. In accordance with a form of embodiment, the solid material core 9 extends in each case from an upper side of the carrier unit 6, which in the stack arrangement is facing towards the number of evaluation units 7, through to a lower side of the carrier unit 6, which is facing away from the number of evaluation units 7.

The thermally conducting filler material 10 may, for example, be a thermally conducting adhesive including aluminum nitride or aluminum oxide. For example, the adhesive may be silicon or epoxy resin-based. In another possible embodiment, the thermally conducting filler may also include solder material.

The gap present between the layers of the stack arrangement may likewise be filled by one or more different underfill materials.

In FIG. 3, the radiation detector module also includes a heat sink 14, where the carrier unit 6 is coupled to the heat sink 14 for heat conduction using a second thermally conducting filler material 11 (e.g., a thermally conducting adhesive). The radiation detector module according to FIG. 2 may likewise be connected to a heat sink 14 in this way.

In accordance with a form of embodiment, the second thermally conducting filler material 11 between the heat sink 14 and the carrier unit 6 is in direct contact with the solid material core 9 in the carrier unit 6 and the heat sink 14.

The heat sink 14 may include a metal (e.g., aluminum). The heat sink may, for example, be provided for a further improved cooling of the radiation detector module by a flow of cooling air that is conveyed along the heat sink 14.

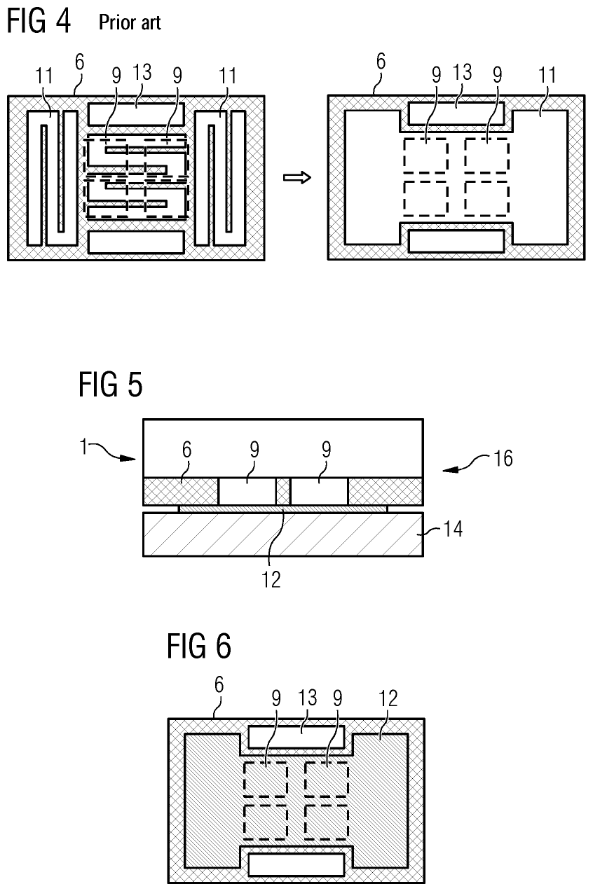

FIG. 4 shows a layer of thermally conducting filler material 11 in accordance with the prior art in a plan view. First, a structure adhesive consisting of a thermally conducting filler material 11 is applied in the form of a bead (left) in a dispensing process. Then carrier unit 6 and heat sink 14 (not shown) are pressed onto one another, and a shape of the heat conducting filler material 11 as shown on the right is produced. This simultaneously serves as an adhesive connection between carrier unit 6 and heat sink 14. Connectors 13 may be free from the thermally conducting filler material 11. The hardening of the structure adhesive typically takes place for a number of minutes or hours at a temperature of between 40° C.-80° C.

FIG. 5 shows an example of a form of embodiment of a radiation detector module 1. The radiation detector module 1 includes a sensor component 16 that also includes a carrier unit 6 and a heat dissipation component 17, for example, that corresponds to a heat sink 14. The sensor component 16 is connected via the carrier unit 6 to the heat sink 14 (e.g., of the heat dissipation component 17) by an RMS connection 12. The sensor areas of the sensor component 16 may be constructed as shown in FIGS. 2 and 3 and include a stack arrangement consisting of a detection layer with a number of converter units 2, 3, 4 configured to convert radiation arriving at the number of converter units 2, 3, 4 into electrical signals together with a number of evaluation units 7 configured to evaluate the electrical signals fed in from the detection layer. In one embodiment, the radiation detector module 1 is configured to be employed in a CT system, as is shown by way of example in FIG. 1.

The carrier unit 6, for example, corresponding to FIGS. 2 and 3, in a surface area that corresponds to a projection of evaluation units 7, in a stack direction, has a solid material core 9 consisting of a thermally conducting material in each case. The solid material core 9 extends over a large part of the respective surface area, and is in contact for heat conduction with the respective evaluation unit 7. The solid material core 9 extends from the upper side of the carrier unit 6 through to the lower side of the carrier unit 6.

FIG. 6 shows an example of an RMS connection 12 in a plan view. Unlike FIG. 4, no meanders are attached, but the form of the attached reactive multilayer system 15 corresponds to the later RMS connection 12. Here too, connectors 13 are left free so that the connectors 13 do not touch the RMS connection 12.

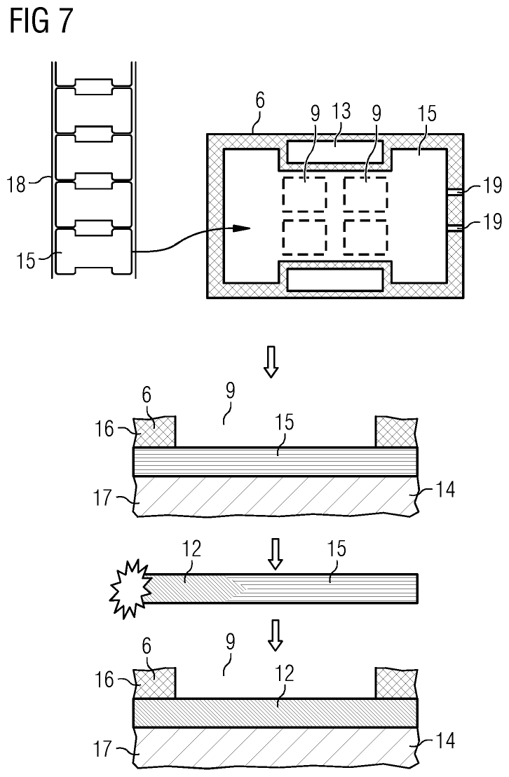

FIG. 7 shows an execution sequence for manufacturing an embodiment of a radiation detector module 1, as is shown, for example, in FIG. 5. Reactive multilayer systems 15 are present, for example, as punched parts in a film and are laid between sensor component 16 (e.g., on the lower side of the carrier unit 6) and heat dissipation component 17 (not shown). They do not absolutely have to be fixed, when sensor component 16 and heat dissipation component 17 are pressed lightly against one another directly after the arrangement of a reactive multilayer system 15, and hold said system.

Two conductor tracks 19 are shown on the right. The two conductor tracks 19 are configured in the carrier unit 6 and reach to the edge of the carrier unit 6. With these conductor tracks 19, a voltage may be applied for activation of the reactive multilayer system 15, using needle contacts, for example.

The upper diagram is a plan view from below of the carrier unit 6. The angle of view changes in the lower illustrations, which show an enlarged side view. Shown in the middle is a part of the reactive multilayer system 15, which is held at the top by the carrier unit 6 with a solid material core 9 and at the bottom by a heat sink 14 as the heat dissipation component 17, and is intended to connect the units.

Shown right at the bottom is the scene with an RMS connection 12, which represents the completed radiation detector module 1.

Depicted between these two diagrams is the activation and the reaction of the reactive multilayer system 15. Using a voltage that is applied via the conductor tracks 19, there is the initial reaction in the reactive multilayer system 15 (e.g., symbolized on the left by the star-shaped flash). The exothermic reaction then runs from left to right through the entire material of the reactive multilayer system 15 and forms the RMS connection 12. The heat arising therefrom for the surrounding material is negligible.

The method described in detail above, and also the computed tomography system 1 shown, merely involve embodiments that may be modified by the person skilled in the art in a wide diversity of ways, without departing from the field of the invention. Further, the use of the indefinite article “a” or “an” does not exclude the features concerned also being able to be present multiple times. Likewise, terms such as “unit” do not exclude the components concerned consisting of a number of subcomponents working together, which, where necessary, may also be spatially distributed. The expression “a number” is to be understood as “at least one”.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for manufacturing a radiation detector module comprising a sensor component and a heat dissipation component, the method comprising:
arranging a reactive multilayer system (RMS) between the sensor component and the heat dissipation component;
bringing together the sensor component and the heat dissipation component; and
activating the reactive multilayer system for creating an RMS connection between the sensor component and the heat dissipation component.

2. The method of claim 1, wherein the heat dissipation component is a heat sink, and the sensor component comprises a carrier unit, and
wherein the reactive multilayer system is arranged between the heat sink and the carrier unit, so that, after the activating, the heat sink and the carrier unit are connected to one another by the RMS connection.

3. The method of claim 2, wherein the carrier unit has a number of solid material cores.

4. The method of claim 1, wherein the reactive multilayer system is attached as an alternating arrangement of lateral layers to a heat sink or a carrier unit (6), preferably by means of sputtering, electrochemical deposition or etching.

5. The method of claim 4, wherein the reactive multilayer system is attached as the alternating arrangement of lateral layers to the heat sink or the carrier unit by sputtering, electrochemical deposition, or etching

6. The method of claim 1, wherein the reactive multilayer system is taken from a film made of reactive multilayer material, and
wherein the reactive multilayer system is pre-fixed to the sensor component, the heat dissipation component, or the sensor component and the heat dissipation component.

7. The method of claim 6, further comprising cutting out or punching out the reactive multilayer system from the fil made of reactive multilayer material.

8. The method of claim 1, wherein the reactive multilayer system is constructed from alternating solder layers and reactive layers, of which materials differ, and
wherein:
the solder layers include a number of materials of a group comprising gold, copper, aluminum, titanium, and metallic glass;
the reactive layers include a material of a group comprising aluminum, titanium, nickel, a-silicon and cobalt; or
a combination thereof.

9. The method of claim 1, wherein conductor tracks are present in the sensor component, the heat dissipation component, or the sensor component and the heat dissipation component, or on the reactive multilayer system, which reach at least up to the sensor component, the heat dissipation component, or the sensor component and the heat dissipation component and by which a voltage is applied for activation of the reactive multilayer system,
wherein parts of the conductor tracks that project beyond the sensor component, the heat dissipation component, or the sensor component and the heat dissipation component are disconnected after the activating.

10. A radiation detector module comprising:
a sensor component; and
a heat dissipation component, the sensor component and the heat dissipation component being connected to one another by a reactive multilayer system (RMS) connection,
wherein a reactive multilayer system is arranged between the sensor component and the heat dissipation component, and
wherein the reactive multilayer system is configured to be activated for creating the RMS connection between the sensor component and the heat dissipation component.

11. The radiation detector module of claim 10, wherein the sensor component comprises a stack arrangement, the stack arrangement comprising:
a detection layer with a number of converter units configured to convert radiation arriving at the number of converter units into electrical signals; and
a number of evaluation units configured to evaluate the electrical signals fed in from the detection layer,
wherein the radiation detector module is configured for detection of x-ray radiation.

12. The radiation detector module of claim 11, wherein the sensor component further comprises:
a carrier unit,
wherein the number of evaluation units is arranged in the stack arrangement between the detection layer and the carrier unit,
wherein the carrier unit includes, in a surface area that corresponds to a projection of a respective evaluation unit of the number of evaluation units in a stack direction, a solid material core made of a thermally conducting material,
wherein the solid material core extends over a part of the respective surface area, and the solid material core is in contact for heat conduction via a thermally conducting filler material with the respective evaluation unit of the number of evaluation units.

13. The radiation detector module of claim 12, wherein the solid material core extends from an upper side of the carrier unit that faces towards the number of evaluation units in the stack arrangement, through to a lower side of the carrier unit that faces away from the number of evaluation units, and
wherein an evaluation unit of the number of evaluation units is attached to the solid material core by the RMS connection.

14. The radiation detector module of claim 12, further comprising a heat sink that is attached to the carrier unit by the RMS connection,
wherein the solid material core of the carrier unit is contacted by the RMS connection on the heat sink.

15. A radiation detector comprising:
a plurality of radiation detector modules arranged next to one another, a radiation detector module of the plurality of radiation detector modules comprising:
a sensor component; and
a heat dissipation component, the sensor component and the heat dissipation component being connected to one another by a reactive multilayer system (RMS) connection, wherein the reactive multilayer system is arranged between the sensor component and the heat dissipation component, and wherein the reactive multilayer system is configured to be activated for creating the RMS connection between the sensor component and the heat dissipation component.

16. An imaging system comprising:
a radiation detector comprising:
a plurality of radiation detector modules arranged next to one another, a radiation detector module of the plurality of radiation detector modules comprising:
a sensor component; and
a heat dissipation component, the sensor component and the heat dissipation component being connected to one another by a reactive multilayer system (RMS) connection, wherein the reactive multilayer system is arranged between the sensor component and the heat dissipation component, and wherein the reactive multilayer system is configured to be activated for creating the RMS connection between the sensor component and the heat dissipation component; and
a radiation source opposite the radiation detector, the radiation source being configured to irradiate the radiation detector.

17. A replacement part for a radiation detector module, the radiation detector module comprising a first sensor component and a heat dissipation component connected to one another by a reactive multilayer system (RMS) connection, wherein a first reactive multilayer system is arranged between the first sensor component and the heat dissipation component, and wherein the first reactive multilayer system is configured to be activated for creating the RMS connection between the first sensor component and the heat dissipation component, the replacement part comprising:
a second sensor component; and
a second reactive multilayer system on a side of the second sensor component that is configured to be connected to the heat dissipation component of the radiation detector module.
